(19) United States
(12) Plant Patent Application Publication
(10) Pub. No.: US 2004/0117886 P1
(43) Pub. Date: Jun. 17, 2004

(54) CHRYSANTHEMUM PLANT NAMED 'CEFREYA'

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 10/316,870

(22) Filed: Dec. 12, 2002

(51) Int. Cl. A01H 5/00

(52) U.S. Cl. PLT/297

(57) ABSTRACT

A chrysanthemum plant named ‘FREYA’ characterized by its medium sized blooms with coral-salmon ray florets and prolific branching; natural season flower date September 1-5; blooming for a period of 5 weeks.

### BACKGROUND OF THE INVENTION

‘FREYA’ is a product of a breeding and selection program for outdoor pot mums (garden mums) which had the objective of creating new chrysanthemum cultivars with a decorative type flower, a natural season flower date around September 1-5; blooming for a period of 5 weeks. The new plant of the present invention comprises a new and distinct cultivar of Chrysanthemum plant ‘FREYA’ is a seedling resulting from the open pollination among groups of chrysanthemum cultivars maintained under the control of the inventor for breeding purposes. The new and distinct cultivar was discovered and selected as one flowering plant by Rob Noodelijk on a cultivated field in Rijsenhout Holland in September 2000. The plant has been asexually reproduced by cuttings in greenhouses at Rijsenhout Holland. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

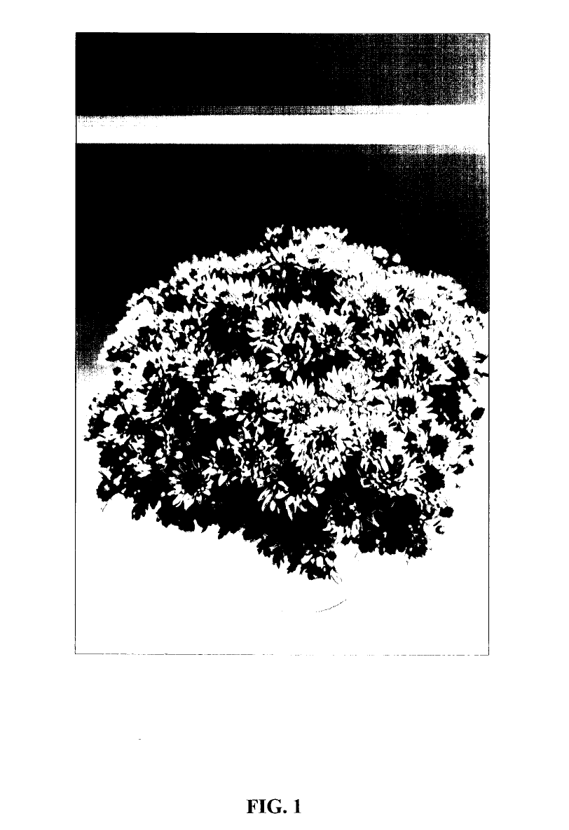

FIG. 2 shows the various stages of bloom of the new cultivar.

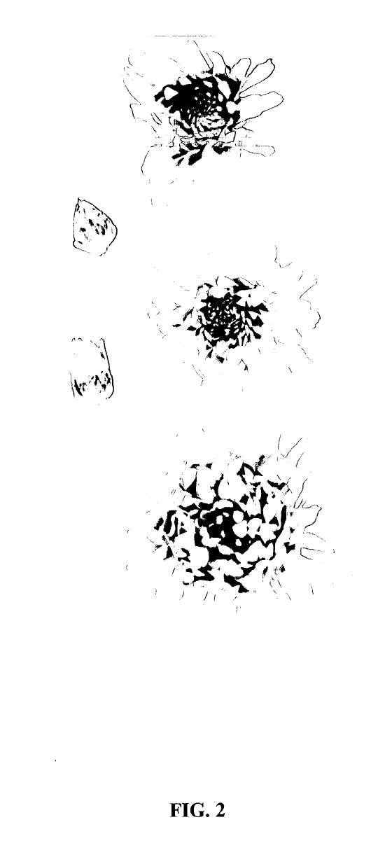

FIG. 3 shows the petiole and foliage of the new cultivar

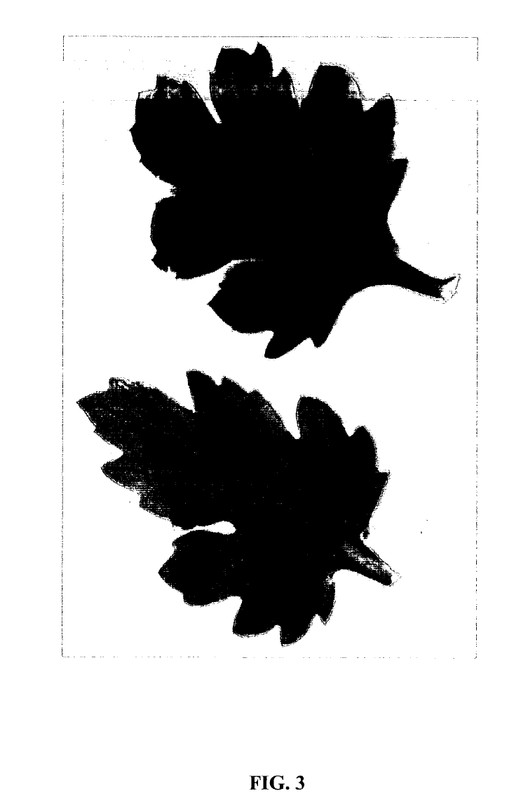

### DESCRIPTION OF THE INVENTION

This new variety of chrysanthemum is of the botanical classification Chrysanthemum morifolium. The observations and measurements were gathered from plants grown out door in Rijsenhout, Holland under natural day length and temperature and planted week 22 in 2000 and 2001 . The natural blooming date of this crop was September 1-5 (week 35). The average height of the plants was 27 cms. No growth retardants were used. No tests were done on disease or insect resistance or susceptibility. No tests were done on cold or drought tolerance. This new variety produces medium sized blooms with coral-salmon ray florets and a red center blooming for a period of 5 weeks.

From the cultivars known to inventor the most similar existing cultivar in comparison to ‘FREYA’ is ‘GRENADINE’ (U.S. Plant Pat. No. 5338). When ‘GRENADINE’ and ‘FREYA’ are being compared the following differences are noticed: The differences of ‘GRENADINE’ and ‘FREYA’ are (1) Flower form. Both ‘FREYA’ and ‘GRENADINE’ has a coral-salmon decorative flower, however the ray-florets differ in form. (2) Natural flowering date. ‘FREYA’ flowers earlier.

The following is a description of the plant and characteristics that distinguish ‘FREYA’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

TABLE 1
Botanical Description of cultivar ‘FREYA’

| | |
|---|---|
| Bud | |
| Size | Medium; cross-section 1.0 cm, height |
| | 1.5 cm. |
| Outside Color | Red 37B |
| Involucral bracts | 2 rows, length 7 mm, width 3 mm |
| Involucral bracts among | Not present |
| disc-florets | |
| Involucral bracts color | Green 138B |
| Bloom | |
| Type | Decorative |
| Height | High; 2.5 cm |
| Size | Medium |
| Fully Expanded | 5.5-6.0 cm |
| Number of blooms per | Approx. 4-6 blooms per branch |
| branch | |
| Performance on the plant | 5 weeks |
| Seeds (if crossed) | Produced in small quantities, ovate grey- |
| | brown 199A, 1½ mm in length. |
| Fragrance | Typical chrysanthemum, slightly |
| Color | |
| Center of the flower | Immature red 37A |
| | Mature red 37C |
| Color of upper surface of | Red 37B |
| the ray-florets | |
| Color of the lower surface | Red 37C |
| of the ray-florets | |
| Tonality from Distance | A garden mum with coral-salmon flowers |
| | and a red disc |
| Color of the upper surface | Red 37C |
| of the ray-florets after | |
| aging of the plant | |
| Ray florets | |
| Texture | Upper and under side smooth |
| Number | 140-150 |
| Cross-section | Flat. |
| Longitudinal axis of | Reflexing to twisted. |
| majority | |
| Length of corolla tube | Short. 0.3 cm |
| Ray-floret margin | Entire |
| Ray-floret length | 2.6-2.8 cm |
| Ray-floret width | 0.6 cm |
| Ratio length/width | High |
| Shape of tip | Rounded |
| Disc florets | None |
| Receptacle shape | Conical raised |
| Reproductive Organs | |
| Stamen | Absent. |
| Pollen | None |
| Styles | Thin |
| Style color | Yellow-green 144A |
| Style Length | 4 mm. |
| Stigmas | Yellow-green 144A |
| Stigma Width | 1 mm. |
| Ovaries | Enclosed in calyx |
| Plant | |
| Form | Grown as spray-type pot mum, outdoor |
| | mounded and round |
| Growth habit | Spreading. |
| Growth rate | Moderate |
| Height | 27 cm |
| Width | 32-34 cm |
| Stem Color | Green 139B |
| Stem Strength | Strong. |
| Stem Brittleness | Present. |
| Stem Anthocyanin | Absent. |
| Coloration | |
| Length of lateral branch | From top to bottom 13-14 cm. |
| Lateral branch color | Green 139B |
| Lateral branch, attach- | Flexible. |
| ment | |
| Branching (average num- | Prolific with 7-8 breaks after pinching |
| ber of lateral branches) | |
| Peduncle length | 6.0 cm |
| Peduncle color | Green 139B |
| Natural season blooming | September 1-5. |
| date | |
| Foliage | |
| Color | Upper side green 139A |
| | Under side green 139C |
| Size | Medium; length 5.0 cm, width 4.0 cm |
| Quantity (number per | 8-9 |
| lateral branch) | |
| Shape | Ovate. |
| Texture upper side | Glabrous |
| Texture under side | Pubescent |
| Venation arrangement | Palmate |
| Shape of the margin | Serrated |
| Shape of Base of Sinus | Round. |
| Between Lateral Lobes | |
| Margin of Sinus Between | Converging. |
| Lateral Lobes | |
| Shape of Base | Truncate |
| Apex | Cuspidate |

TABLE 2
Differences with the comparison varieties

| | ‘FREYA’ | ‘GRENADINE’ |
|---|---|---|
| Longitudinal axis of ray-florets | Reflexing to twisted | Incurved |
| Cross section of ray-florets | Flat | Concave |
| Natural flowering date | September 1-5 | September 17-25 |

I claim:

1. A new and distinct variety of chrysanthemum plant as described and illustrated.
